(19) United States
(12) Patent Application Publication
(10) Pub. No.: US 2025/0176928 A1
(43) Pub. Date: Jun. 5, 2025

(54) DYNAMIC IMAGING SYSTEM, IMAGING CONTROL METHOD, AND STORAGE MEDIUM

(71) Applicant: Konica Minolta, Inc., Tokyo (JP)

(72) Inventor: Kojiro ONO, Narita-shi (JP)

(21) Appl. No.: 18/961,845

(22) Filed: Nov. 27, 2024

(30) Foreign Application Priority Data
Dec. 1, 2023 (JP) 2023-203967

(51) Int. Cl. A61B 6/00; G01T 1/17

(52) U.S. Cl. CPC A61B 6/542; A61B 6/5294; G01T 1/17

(57) ABSTRACT

A dynamic imaging system includes: a radiation source, a radiation detector, and a hardware processor. The dynamic imaging system is configured to perform dynamic imaging of obtaining a dynamic image constituted of multiple frames by irradiating a subject with radiation by the radiation source and detecting the radiation passing through the subject by the radiation detector. The hardware processor controls the radiation source and the radiation detector to operate in an intermittent imaging mode in which dynamic imaging is performed multiple times from one time of imaging start to one time of imaging end, based on a specified imaging period and a specified imaging suspension period.

### CROSS-REFERENCE TO RELATED APPLICATIONS

The entire disclosure of Japanese Patent Application No. 2023-203967 filed on Dec. 1, 2023 is incorporated herein by reference in its entirety.

### TECHNICAL FIELD

The present invention relates to a dynamic imaging system, an imaging control method, and a storage medium.

### DESCRIPTION OF THE RELATED ART

There is known a radiographic imaging system for performing dynamic imaging of a subject in a place of a hospital other than an imaging room (e.g., a hospital room, an operation room) (for example, refer to Japanese Unexamined Patent Publication No. 2019-5073). Such a radiographic imaging system is convenient in that dynamic imaging can be performed for a patient difficult to move to an imaging room, such as a seriously injured person.

However, in imaging outside the imaging room, the total dose per imaging is limited from the viewpoint of risk management, and X-ray pulse irradiation cannot be performed for a long time. Since imaging cannot be performed for a long time, images at necessary timings may not be obtained when, for example, a user wishes to check the state inside the body by dynamic imaging during treatment or after the patient takes a contrast medium.

An object of the present invention is to enable acquisition of a dynamic image for a required period of time while suppressing an increase of exposure dose.

### SUMMARY OF THE INVENTION

In order to solve the above problem, according to an aspect of the present invention, there is provided a dynamic imaging system including: a radiation source; a radiation detector; and a hardware processor, wherein the dynamic imaging system is configured to perform dynamic imaging of obtaining a dynamic image constituted of multiple frames by irradiating a subject with radiation by the radiation source and detecting the radiation passing through the subject by the radiation detector; and the hardware processor controls the radiation source and the radiation detector to operate in an intermittent imaging mode in which dynamic imaging is performed multiple times from one time of imaging start to one time of imaging end, based on a specified imaging period and a specified imaging suspension period.

According to another aspect of the present invention, there is provided an imaging control method for a dynamic imaging system that includes a radiation source and a radiation detector and that is configured to perform dynamic imaging of obtaining a dynamic image constituted of multiple frames by irradiating a subject with radiation by the radiation source and detecting the radiation passing through the subject by the radiation detector, the method including: controlling the radiation source and the radiation detector to operate in an intermittent imaging mode in which dynamic imaging is performed multiple times from one time of imaging start to one time of imaging end, based on a specified imaging period and a specified imaging suspension period.

According to another aspect of the present invention, there is provided a non-transitory computer-readable storage medium storing a program for a computer of a dynamic imaging system that includes a radiation source and a radiation detector and that is configured to perform dynamic imaging of obtaining a dynamic image constituted of multiple frames by irradiating a subject with radiation by the radiation source and detecting the radiation passing through the subject by the radiation detector, the program causing the computer to: control the radiation source and the radiation detector to operate in an intermittent imaging mode in which dynamic imaging is performed multiple times from one time of imaging start to one time of imaging end, based on a specified imaging period and a specified imaging suspension period.

### BRIEF DESCRIPTION OF THE DRAWINGS

The advantages and features provided by one or more embodiments of the invention will become more fully understood from the detailed description given hereinbelow and the appended dmwings which are given by way of illustration only, and thus are not intended as a definition of the limits of the present invention.

FIG. 1 shows an example of an overall configuration of an in-hospital system including a dynamic imaging system;

FIG. 2 is a block diagram of a functional configuration of a main body;

FIG. 3 is a schematic diagram showing pulse irradiation in a normal mode;

FIG. 4 is a schematic diagram showing pulse irradiation in an intermittent imaging mode;

FIG. 5 shows an example of an imaging screen;

FIG. 6 is a flowchart of an intermittent imaging mode process A executed by the controller of FIG. 1;

FIG. 7 shows an example of the imaging screen in which a transferred frame is displayed in an image display section;

FIG. 8 is a schematic view of a state in which multiple dynamic images obtained by intermittent imaging are connected into a dynamic image of one series;

FIG. 9 shows an example of a specifying screen;

FIG. 10 is a flowchart of an intermittent imaging mode process B executed by the controller of FIG. 1;

FIG. 11 is a schematic diagram of dynamic images obtained by intermittent imaging and each generated as one dynamic image of one series; and

FIG. 12 shows an example of displaying an obtained dynamic image next to the other image in the image display section such that the images can be compared with each other.

### DETAILED DESCRIPTION

Hereinafter, embodiments of the present invention are described below with reference to the drawings. However, the embodiments described below have various limitations which are technically preferable for carrying out the present invention. Therefore, the technical scope of the present invention is not limited to the following embodiments and illustrated examples.

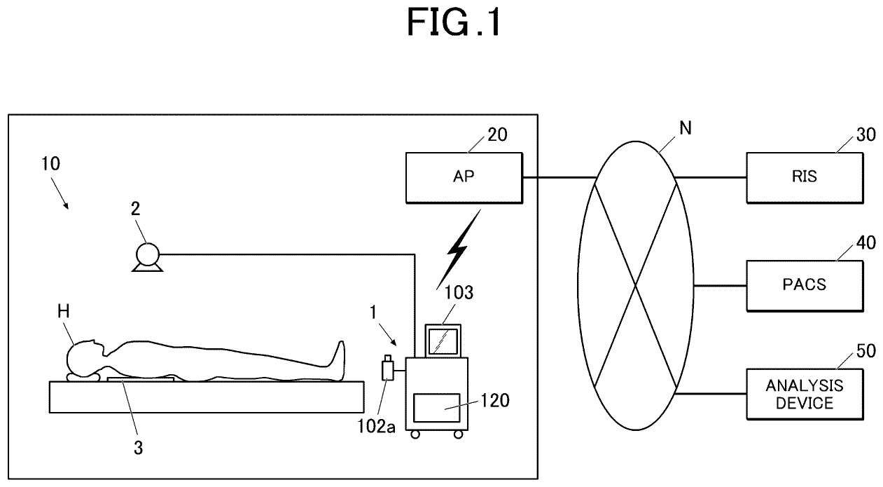

FIG. 1 shows an example of the overall configuration of an in-hospital system including a dynamic imaging system 10 in the present embodiment. The dynamic imaging system 10 is, for example, a system for performing dynamic imaging in a round visit for a patient who is difficult to move. The dynamic imaging system 10 includes a main body 1, a radiation source 2, and a FPD 3. The main body 1 has wheels and is configured as a movable medical cart. Note that the dynamic imaging system 10 may be portable without wheels. The main body 1 is connected to a communication network N such as an in-hospital local area network (LAN) via a wireless access point (AP) 20 installed in a hospital. The main body 1 can transmit and receive data to and from external devices, such as a radiology information system (RIS) 30,
a picture archiving and communication system (PACS) 40, and an analysis device 50, via the communication network N.

The dynamic imaging system 10 performs static imaging or dynamic imaging of a subject H by irradiating the subject H with radiation from the radiation source 2 in a state where the FPD 3 is disposed opposite the radiation source 2 with the subject H inbetween. In the present embodiment, dynamic imaging refers to acquiring multiple images of a subject by repeatedly irradiating the subject with pulsed radiation, such as X-rays, at predetermined time intervals (pulse irradiation). A series of images obtained by dynamic imaging is called a dynamic image. Each of the images constituting a dynamic image is called a frame.

Note that dynamic imaging includes moving image capturing but does not include capturing still images while displaying a moving image. Further, examples of a dynamic image includes a moving image but does not include still images captured while displaying a moving image.

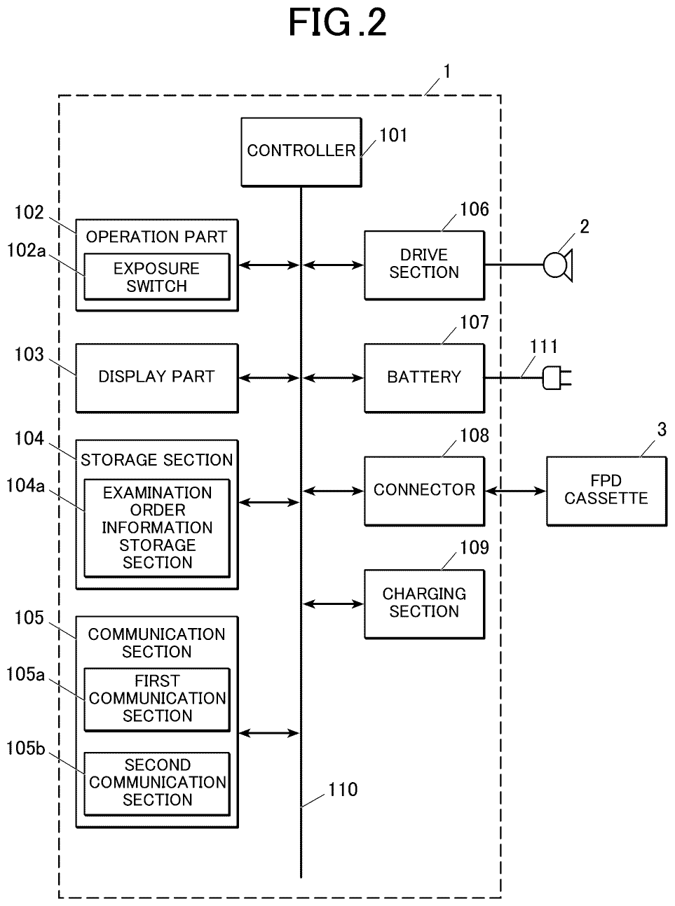

FIG. 2 is a block diagram illustrating the functional configuration of the main body 1.

The main body 1 serves as a console (imaging control device). As shown in FIG. 2, the main body 1 includes a controller 101 (hardware processor), an operation part 102, a display part 103, a storage section 104, a communication section 105, a drive section 106, a battery 107, a connector 108, and a charging section 109. The parts of the main body 1 are connected by a bus 110.

The controller 101 includes a central processing unit (CPU) and a random access memory (RAM). In response to an input from the operation part 102, the controller 101 reads a system program or various processing programs stored in the storage section 104, loads the program in the RAM, and executes various processes in accordance with the loaded program.

The operation part 102 includes a touch screen or the like in which transparent electrodes are arranged in a lattice shape so as to cover the surface of the display part 103. The touch screen detects the position pressed by a finger, a touch pen, or the like, and inputs position information as operation information to the controller 101.

The operation part 102 includes an exposure switch 102a. The exposure switch 102a is a switch for the user to instruct irradiation with the radiation source 2.

The display part 103 is constituted of a monitor such as a liquid crystal display (LCD) or a cathode ray tube (CRT). The display part 103 displays screens in accordance with an instruction of a display signal input from the controller 101.

The storage section 104 includes a nonvolatile semiconductor memory, a hard disk, or the like. The storage section 104 stores various programs to be executed by the controller 101, parameters required to execute processing by the programs, and data such as processing results.

Furthermore, in the present embodiment, the storage section 104 includes an examination-order-information storage section 104a.

The examination-order-information storage section 104a stores examination order information acquired from the RIS 30. The examination order information includes patient information and examination information. The patient information includes the patient ID, name, sex, age, and the hospital room (ward) of the patient to be examined. The examination information includes the examination ID, examination date, and the imaging order for each imaging to be performed in the examination. The imaging order includes an imaging part, an imaging direction, and the classification of still imaging or dynamic imaging.

The storage section 104 includes a temporary storage area (not shown) for temporarily storing medical images transferred from the FPD 3. Furthermore, the storage section 104 includes an image storage area (not illustrated) for storing medical images transferred from the FPD 3 for a certain period of time in association with supplementary information.

The communication section 105 includes a first communication section 105a and a second communication section 105b. The first communication section 105a performs data transmission and reception to and from the FPD 3 by wired communication or wireless communication. The second communication section 105b performs data transmission and reception to and from an external device, such as the RIS 30 or the PACS 40 connected to the communication network N via the wireless access point 20. The second communication section 105b functions as an outputter.

The drive section 106 is a circuit that drives the tube of the radiation source 2. The drive section 106 and the radiation source 2 are connected to each other via a cable.

The battery 107 supplies electric power to each part of the main body 1 and the radiation source 2. The battery 107 can be charged externally via an AC cable 111.

The connector 108 is provided inside a storage part 120 and is electrically connected to the FPD 3 stored in the storage part 120.

The charging section 109 charges the FPD 3 connected via the connector 108 with the power supplied by the battery 107 under the control of the controller 101.

The radiation source 2 is driven by the drive section 106 to irradiate the subject H with radiation (X-rays). In dynamic imaging, the radiation source 2 repeatedly irradiates the subject H with pulsed radiation at predetermined time intervals, for example.

The FPD 3 is a portable radiographic detector that is compatible with still imaging and dynamic imaging. The FPD 3 includes, for example, radiation detection elements arranged two-dimensionally on a glass substrate. Each radiation detection element is composed of a semiconductor image sensor, such as a photodiode. The radiation detection element detects, based on the radiation intensity, radiation emitted by the radiation source 2 and transmitted through at least the subject H, converts the detected radiation into an electric signal, and accumulates the electric signal. For example, each of the radiation detection elements is connected to a switching unit, such as a thin film transistor (TFT). The switching unit controls the accumulation and reading of the electrical signal, and image data is acquired. There are an indirect conversion type FPD that converts radiation to electrical signals by photoelectric conversion elements via a scintillator and a direct conversion type FPD that directly converts radiation to electrical signals. Either type may be used.

In the present embodiment, the description will be given on the assumption that the FPD 3 is a so-called self-detection type FPD. That is, the FPD 3 has an automatic detection mode function of automatically detecting irradiation. In dynamic imaging, in response to detecting start of irradiation, the FPD 3 performs imaging (accumulation and reading) at a preset frame rate. The frames obtained by imaging are sequentially transferred to the main body 1 by a communication section (not shown). Note that the FPD 3 is not limited to the self-detection type.

The RIS 30 generates and stores examination order information. The RIS 30 transmits the generated examination order information to the main body 1 of the dynamic imaging system 10 via the communication network N.

The PACS 40 stores and manages medical images generated by a modality such as the dynamic imaging system 10 in association with supplementary information (patient information and examination information) of the medical images. The medical images include a still image and a dynamic image.

The analysis device 50 analyzes a medical image generated by a modality such as the dynamic imaging system 10 and outputs an analysis result.

### (Operation of Dynamic Imaging System)

Next, an imaging operation of the dynamic imaging system 10 in the intermittent imaging mode according to the present embodiment will be described.

In known dynamic imaging, pulse irradiation by the radiation source 2 is continuously performed from the start to the end of imaging to acquire a dynamic image consisting of a series of frames. However, in performing imaging outside the imaging room, the total dose that can be applied from the start to the end of imaging is limited from the viewpoint of risk management. Therefore, pulse irradiation cannot be performed for a long time. Since the imaging cannot be performed for a long time, images at necessary timings may not be obtained in a case where dynamic imaging is performed to check the state of a procedure of catheter insertion or to check the state inside the body immediately after a contrast medium is taken.

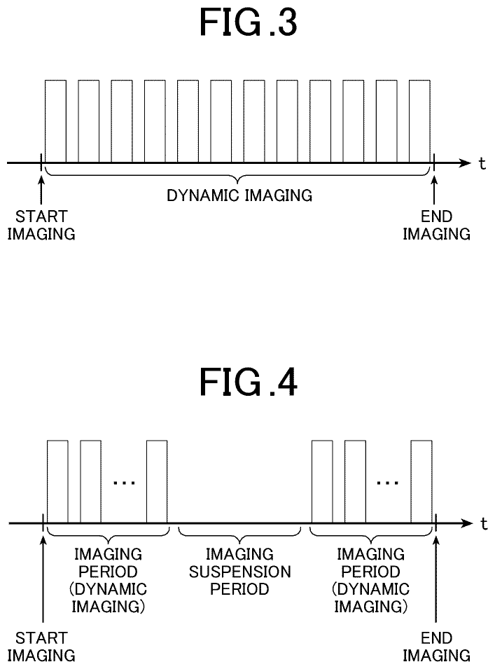

To deal with the above issue, the dynamic imaging system 10 of the present embodiment is configured to perform dynamic imaging in a normal mode and an intermittent imaging mode. FIG. 3 shows a schematic diagram of pulse irradiation in the normal mode, and FIG. 4 shows a schematic diagram of pulse irradiation in the intermittent imaging mode.

In the normal mode, dynamic imaging is performed in the same way as the conventional art. That is, in the normal mode, pulse irradiation by the radiation source 2 is continuously performed from the start to the end of the imaging, as shown in FIG. 3. In the normal mode, one dynamic image consisting of a series of frames is obtained. In the intermittent imaging mode, dynamic imaging, in which pulse irradiation by the radiation source 2 is continuously performed, is intermittently performed multiple times from the start to the end of one time of imaging, based on a specified imaging period and a specified imaging suspension period, as shown in FIG. 4. In the intermittent imaging mode, multiple dynamic images, each of which consists of a series of frames, are obtained. Each execution of dynamic imaging in the intermittent imaging mode is called intermittent imaging.

In the present embodiment, in the normal mode, the radiation source 2 continues pulse irradiation from the start to the end of imaging. In the intermittent imaging mode, during the period from the start to the end of imaging, the radiation source 2 performs pulse irradiation during the specified imaging period; and during the specified imaging suspension period, the radiation source 2 stops pulse irradiation and stands by while rotating the anode.

In the normal mode, after the imaging start is notified, the FPD 3 detects radiation and starts accumulation and reading of charges corresponding to the radiation. When the FPD 3 does not detect radiation for a predetermined period or longer, the FPD 3 determines that the imaging ends and ends the accumulation and reading of charges. In the intermittent imaging mode, after the imaging start is notified, the FPD 3 detects radiation and starts accumulation and reading of charges corresponding to the radiation. Thereafter, even if the FPD 3 does not detect radiation for a predetermined period or longer, the FPD 3 recognizes that imaging is in progress until the imaging end is notified and continues accumulation and reading of charges corresponding to the detected radiation.

In dynamic imaging, an imaging executor (user) manipulates the operation part 102 of the main body 1 to display an examination order list screen. The controller 101 of the main body 1 causes the display part 103 to display the examination order list screen (not illustrated), based on the examination order information stored in the examination-order-information storage section 104a. The user selects an examination (imaging target) in which dynamic imaging is to be performed from the examination order list screen by manipulating the operation part 102. The controller 101 causes the display part 103 to display the imaging screen 130 corresponding to the selected examination.

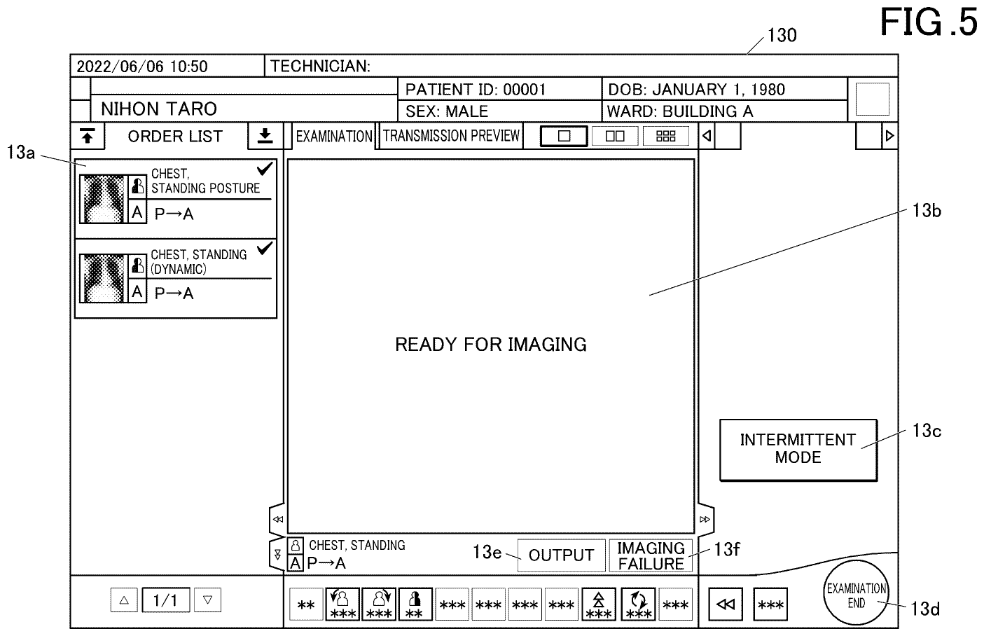

FIG. 5 shows an example of the imaging screen 130. As shown in FIG. 5, the imaging screen 130 includes an imaging order list 13a, an image display section 13b, an intermittent mode button 13c, an examination end button 13d, an output button 13e, and an imaging failure button 13f.

The imaging order list 13a is a list of imaging orders included in the examination order information of the selected examination. The imaging order includes an imaging part, an imaging direction, and the classification of still imaging or dynamic imaging.

The image display section 13b is a section for displaying an obtained image. Before imaging is performed, the image display section 13b displays information indicating the state of the system, such as “Waiting for exposure permission” or “Ready for imaging”.

The intermittent mode button 13c is a button for giving an instruction to start and end imaging in the intermittent imaging mode. When the imaging order of dynamic imaging is selected, the intermittent mode button 13c is activated and can be pressed.

The output button 13e is a button for instructing output of an obtained image to the PACS 40 and/or the analysis device 50.

The imaging failure button 13f is a button for giving an instruction to discard an obtained image without outputting the image.

The examination end button 13d is a button for giving an instruction to end the examination.

The user selects an imaging order from the imaging order list 13a on the imaging screen 130 by manipulating the operation part 102 and prepares for imaging by doing positioning and so forth.

In the main body 1, when the imaging order of dynamic imaging is selected by the manipulation of the operation part 102, the controller 101 displays “Waiting for exposure permission” in the image display section 13b and activates the intermittent mode button 13c. The controller 101 causes the radiation source 2 and the FPD 3 to prepare for imaging. For example, the controller 101 sets irradiation conditions corresponding to the selected imaging order to the drive section 106 and causes the drive section 106 to activate the radiation source 2 and keep the radiation source 2 at a standby state. Further, the controller 101 transmits image reading conditions corresponding to the selected imaging order to the FPD 3 via the first communication section 105a and causes the FPD 3 to prepare for imaging (e.g., to perform reset processing). When the reset processing ends, the FPD 3 proceeds to the automatic detection mode.

When notified by the drive section 106 and the FPD 3 that the preparations for imaging have been completed, the controller 101 causes the image display section 13b to display “Ready for imaging”. When the intermittent mode button 13c is pressed, the controller 101 instructs the drive section 106 and the FPD 3 to start imaging in the intermittent imaging mode.

The controller 101 starts the intermittent imaging mode process A and performs intermittent imaging multiple times in response to the exposure switch 102a being pressed by the user.

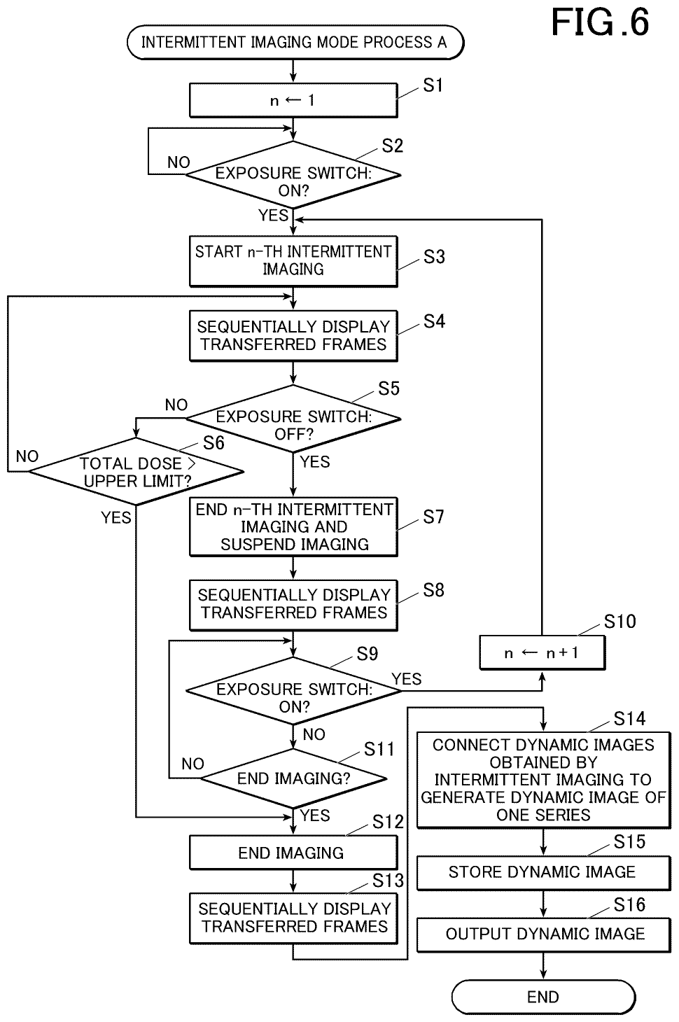

FIG. 6 is a flowchart of the intermittent imaging mode process A in the present embodiment. The intermittent imaging mode process A is executed by the controller 101 in cooperation with a program stored in the storage section 104.

In the intermittent imaging mode process A, the user specifies the imaging period and the imaging suspension period of intermittent imaging by manipulating the exposure switch 102a. For example, the user can instruct the start of intermittent imaging by turning on the exposure switch 102a. The user can instruct the suspension of imaging by turning off the exposure switch 102a. That is, the imaging period of intermittent imaging is between the ON of the exposure switch 102a to the OFF of the exposure switch 102a; and the imaging suspension period is between the OFF of the exposure switch 102a to the ON of the exposure switch 102a. The ON of the exposure switch 102a is, for example, pressing (fully pressing) the exposure switch 102a. The OFF of the exposure switch 102a is, for example, releasing the exposure switch 102a.

The imaging period is a period during which pulse irradiation is performed by the radiation source 2. The imaging suspension period is a period during which pulse irradiation by the radiation source 2 is suspended.

In the intermittent imaging mode A, first, the controller 101 sets a variable n to 1 (step S1).

Next, the controller 101 waits for the exposure switch 102a to be turned on (step S2). When the exposure switch 102a is turned on (step S2; YES), the controller 101 causes the drive section 106 to cause the radiation source 2 to start pulse irradiation and to start the n-th intermittent imaging (step S3). For example, the controller 101 causes the drive section 106 and the radiation source 2 to perform pulse irradiation at a frame rate specified in the irradiation conditions.

When the FPD 3 detects radiation, the FPD 3 repeats a process for obtaining a frame with an accumulation time and a reading time corresponding to a frame rate specified in the image reading conditions. The FPD 3 adds, to the obtained frame, the frame number indicating the imaging order and the identification number of the intermittent imaging and transfers the frame to the main body 1.

The controller 101 receives, via the first communication section 105a, the frames transferred from the FPD 3 and sequentially displays the frames in the image display section 13b (step S4).

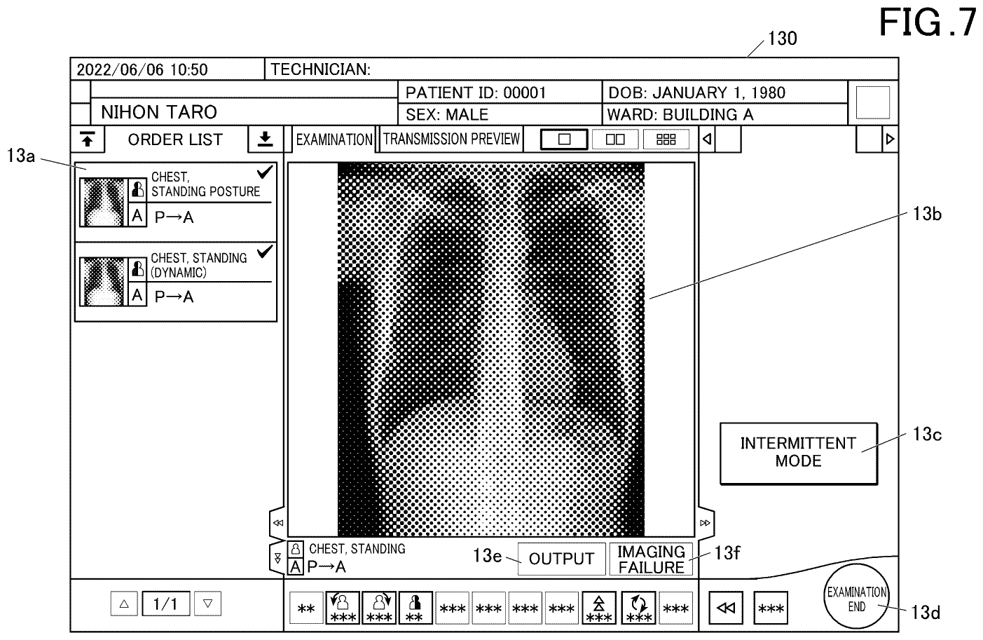

FIG. 7 shows an example of the imaging screen 130 in which the transferred frame is displayed in the image display section 13b.

Here, the controller 101 stores the transferred frames in a temporary storage area of the storage section 104. It is preferable that the controller 101 perform offset correction, gain correction, gradation correction, and the like on the frames before displaying the frames. To simplify and speed up the internal process of offset correction, an image for offset correction (dark image) may be obtained in the first intermittent imaging, and offset correction may be performed using the same image for offset correction during the imaging period. Of course, the offset correction image may be obtained for each intermittent imaging.

Next, the controller 101 determines whether the exposure switch 102a has been turned off or not (step S5).

When determining that the exposure switch 102a has not been turned off (step S5; NO), the controller 101 determines whether the total dose after the imaging start has exceeded a predetermined upper limit value (step S6). When determining that the total dose has not exceeded the upper limit value (step S6: NO), the controller 101 returns to step S4. That is, the controller 101 continues the n-th intermittent imaging, receives frames transferred from the FPD 3 via the first communication section 105a, and sequentially displays the frames in the image display section 13b.

If the controller 101 determines that the total dose of irradiation has exceeded the upper limit value (step S6; YES), the controller 130 proceeds to step S12.

On the other hand, when determining that the exposure switch 102a has been turned off (step S5; YES), the controller 101 ends the n-th intermittent imaging and suspends the imaging (step S7).

That is, the controller 101 causes the drive section 106 to suspend (temporarily stop) irradiation by the radiation source 2. When radiation is not detected, the FPD 3 transmits untransmitted frames while waiting for the next detection of radiation. While radiation is not detected, the FPD 3 may continue accumulation and reading but discards images obtained during this period and does not transfer the images to the main body 1.

While the imaging is suspended, the controller 101 keeps receiving the frames transferred from the FPD 3 via the first communication section 105a and causes the image display section 13b to sequentially display the frames (step S8).

While the imaging is suspended, in response to receiving the last frame in the intermittent imaging immediately before the suspension via the first communication section 105a, the controller 101 causes the image display section 13b to display the last frame and keep displaying the last frame. Instead of displaying the last frame, the controller 101 may extract a characteristic frame from the group of the intermittent imaging immediately before the suspension and display the extracted frame in the image display section 13b, in accordance with a predetermined setting or definition. For example, the controller 101 may extract the last frame in which the movement of the catheter is stopped, based on the detection of the catheter by image analysis, and may display the frame in the image display section 13b. For another example, only during imaging is suspended, the controller 101 may perform image processing on the last frame, such as scattered radiation correction processing or frequency enhancement processing, in accordance with a predetermined setting, and display the processed last frame instead of displaying the unprocessed original last frame.

While imaging is suspended, it is preferable that the controller 101 display notification information, such as an icon or a mark, indicating that the image displayed in the image display section 13b is not a real-time image. For example, if the controller 101 can recognize that the imaging is suspended according to the OFF state of the exposure switch 102a or according to the state where the suspension period has elapsed for a predetermine period, it is preferable that the controller 101 indicate that imaging is suspended by showing an overlay on the final frame displayed in the image display section 13b.

Next, the controller 101 determines whether the exposure switch 102a has been turned on or not (step S9).

If the controller 101 determines that the exposure switch has been turned on (step S9: YES), the controller 101 increments the variable n (step S10) and returns to step S3. That is, the controller 101 starts the next intermittent imaging and sequentially displays frames obtained by the imaging in the image display section 13b.

On the other hand, if the controller 101 determines that the exposure switch has not been turned on (step S9; NO), the controller 130 determines whether the end of imaging has been instructed (step S11).

Herein, when the intermittent mode button 13c on the imaging screen 130 is pressed again, the controller 101 determines that the end of imaging has been instructed.

When determining that the end of imaging has not been instructed (step S11; NO), the controller 101 returns to step S9.

When determining that the end of imaging has been instructed (step S11; YES), the controller 101 proceeds to step S12.

In step S12, the controller 101 notifies the drive section 106 and the FPD 3 of the end of imaging and ends the imaging (step S12).

The controller 101 keeps receiving frames forwarded from the FPD 3 via the first communication section 105a and causes the image display section 13b to sequentially display the frames (step S13).

After all the frames obtained by the imaging are displayed, the controller 101 connects the multiple dynamic images obtained by multiple times of intermittent imaging to generate a dynamic image of one series (step S14).

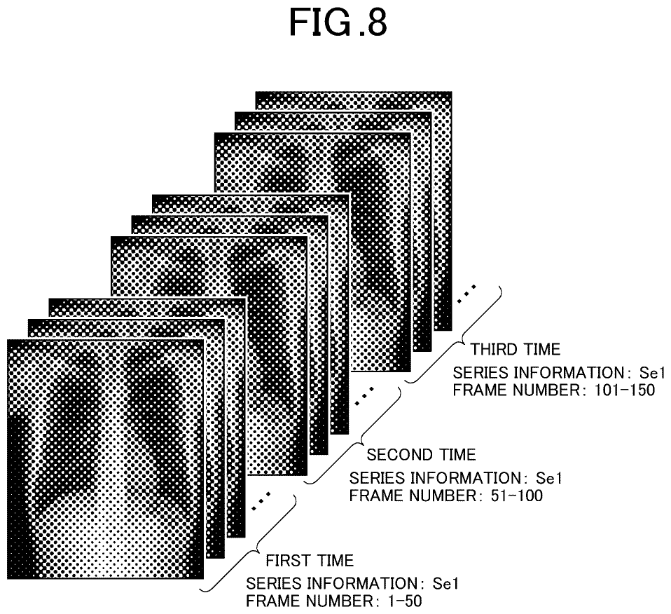

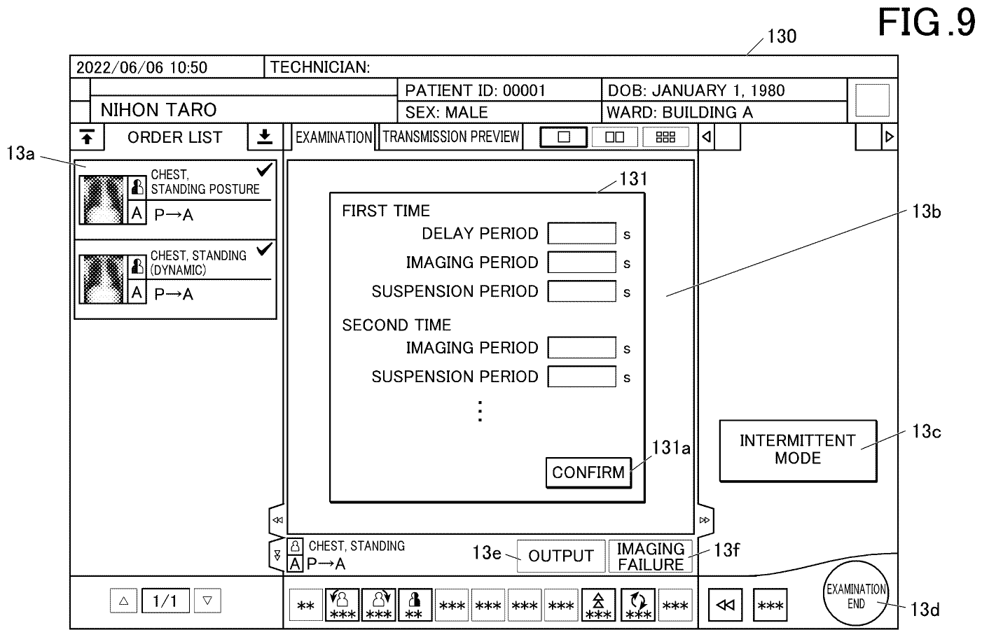

Step S14 is described, based on an example case where three times of intermittent imaging are performed from the start to the end of one time of imaging. In the example case, the frames of a dynamic image in the first intermittent imaging have frame numbers 1 to 50; the frames of a dynamic image in the second intermittent imaging have frame numbers 51 to 100; and the frames of a dynamic image in the third intermittent imaging have frame numbers 101 to 150. In the case, as illustrated in FIG. 8, the controller 101 attaches the same series information (e.g., Se1) to each of the frames corresponding to frame numbers 1 to 150 to generate a dynamic image of one series. The series information is, for example, a series ID for identifying a series in the same examination. By attaching the same series information to the frames, the controller 101 can associate multiple dynamic images obtained by multiple times of intermittent imaging as a dynamic image of the same series in the same examination. In addition to the series information, the controller 101 adds a frame number, patient information, and examination information to each of the frames.

In connecting multiple dynamic images, the controller 101 may insert a summary image, which is a frame with the description of the imaging conditions, into a connection portion (joint) between the dynamic images. The summary image includes, for example, imaging conditions of the intermittent imaging in which the frame group immediately before the connecting portion is imaged (e.g., delay period, imaging time, the tube voltage, and the mAs value) and the imaging interruption period. The summary image, which is inserted in the connection portion, may include a list of imaging conditions of the respective times of intermittent imaging. Alternatively, one predetermined frame may be inserted in the connection portion, or predetermined frames of the number corresponding to the imaging suspension period may be inserted. The predetermined frame(s) is, for example, a copy of the last frame, an entirely black frame, or a frame of a predetermined pattern. Such a frame allows the user to easily recognize where the connection portion is.

Dynamic images may be connected without inserting a summary frame or a predetermined frame in the connection portion, and a summary image showing the imaging conditions of the intermittent imaging may be inserted at the end of a series of frames. In such a case, texts such as “This is the last frame of n-th intermittent imaging” may be overlaid on the last frame of each intermittent imaging so that the connecting portion is recognizable, for example.

For another example, on each of the frames, information indicating the frame number and the intermittent imaging may be overlaid, such as “1-1” for the first frame of the first intermittent imaging and “2-5” for the fifth frame of the second intermittent imaging.

The information indicating which intermittent imaging the frame belongs to or the information indicating what the frame number is and which intermittent imaging the frame belongs to may be added to supplementary information or header information of the image, instead of being overlaid on the image. By adding information indicating which intermittent imaging the frame belongs to, it is possible to display frames from the start of each time of intermittent imaging or display frames based on the unit of intermittent imaging. For example, in a case of intermittently imaging a swallowing motion, the frame corresponding to the swallowing timing is firstly viewed. The user can firstly check a dynamic image of intermittent imaging including the swallowing timing in the later stage and then check dynamic images sequentially obtained in multiple times of intermittent imaging from the imaging start.

Next, the controller 101 associates the generated dynamic image with the supplementary information and stores the dynamic image in the image storage region of the storage section 104 (step S15).

The controller 101 also transmits (outputs) the generated dynamic image to the PACS 40 and/or the analysis device 50 via the second communication section 105b (step S16). The controller 101 then ends the intermittent imaging mode process A.

In the above embodiment, the imaging start and the imaging end are instructed by pressing the intermittent mode button 13c. Instead, the imaging start and the imaging end may be instructed by manipulating the exposure switch 102a.

For example, in a case where the exposure switch 102a is a two-stage switch that can be half-pressed and fully-pressed, the first full press of the exposure switch 102a corresponds to an instruction to start imaging and to start the first intermittent imaging. The change from the fully-pressed state to the half-pressed state of the exposure switch 102a corresponds to an instruction to suspend imaging. The return from the half-pressed state to the fully-pressed state of the exposure switch 102a corresponds to an instruction to start the next intermittent imaging. The release of the exposure switch 102a corresponds to an instruction to end the imaging.

The controller 101 may automatically determine to end imaging when any of the following conditions (1) to (5) is satisfied.
(1) The total imaging time from the imaging start has reached a predetermined imaging time.

Thus, the total of actual imaging time by intermittent imaging does not exceed a predetermined imaging time.
(2) The total irradiation dose has reached a predetermined irradiation dose, as described above.
(3) The total of the imaging period and the imaging suspension period has reached a predetermined period.

For example, an upper limit is set to three minutes, and imaging and pausing can be performed for any number of times during three minutes.
(4) The suspension period of intermittent imaging has reached a predetermined period.

Thus, when the suspension period is equal to or longer than the predetermined period, the imaging can be ended in view of imaging quality, and the reset processing can be performed.
(5) The number of times of intermittent imaging has reached a predetermined number.

A technician may not intuitively recognize the period and the dose. By managing intermittent imaging based on the number of times the intermittent imaging can be performed, the technician can easily recognize the timing to end the imaging.

The setting as to which of the above-described conditions (1) to (5) is applied to end imaging may be stored in the storage section 104 beforehand, or the user may specify the condition among the conditions (1) to (5) on a control panel or a dialog of the imaging screen 130 before imaging is performed. During imaging, the controller 101 may display information on the current period of time, dose, and/or the number of times the intermittent imaging has been performed on the imaging screen 130 or notify the information by sound. Thus, the controller 101 may notify the user of how long or how many times imaging can be performed.

### Second Embodiment

Next, a second embodiment of the present invention will be described.

Since the configuration of the dynamic imaging system 10 according to the second embodiment is the same as the one described in the first embodiment, the description thereof is omitted. The operation in the second embodiment is described below.

In response to the intermittent mode button 13c being pressed on the imaging screen 130, the controller 101 displays a specifying screen 131 in a pop-up, as illustrated in FIG. 9. Before imaging is started, the specifying screen 131 allows the user to specify a delay period between the imaging start and the first intermittent imaging, an imaging period for each intermittent imaging, and an imaging suspension period. By specifying the delay period, the imaging period, and the imaging suspension period on the specifying screen 131, the user can specify the imaging period and the imaging suspension period of each intermittent imaging.

When the user performs inputs on the specifying screen 131 by manipulating the operation part 102, the controller 101 calculates the total dose of this series of dynamic imaging, based on the input imaging period and irradiation conditions for each intermittent imaging. If the calculated total dose exceeds a predetermined upper limit value, the controller 101 displays, for example, a warning such as “The dose exceeds the upper limit” on the specifying screen 131 to prompt the user to change the imaging period or the number of times of imaging. When the user makes inputs so that the total dose is equal to or below the upper limit value and presses the “confirm” button 131a, the controller 101 stores (sets) the specified information in a predetermined region of the storage section 104. The controller 101 also instructs the drive section 106 and the FPD 3 to start imaging in the intermittent imaging mode. The controller 101 starts the intermittent imaging mode processing B and performs control such that intermittent imaging is performed multiple times in accordance with the specified delay period, imaging period, and imaging suspension period.

The user may be allowed to specify imaging conditions (e.g., the tube voltage and the mAs value) for each intermittent imaging on the specifying screen 131.

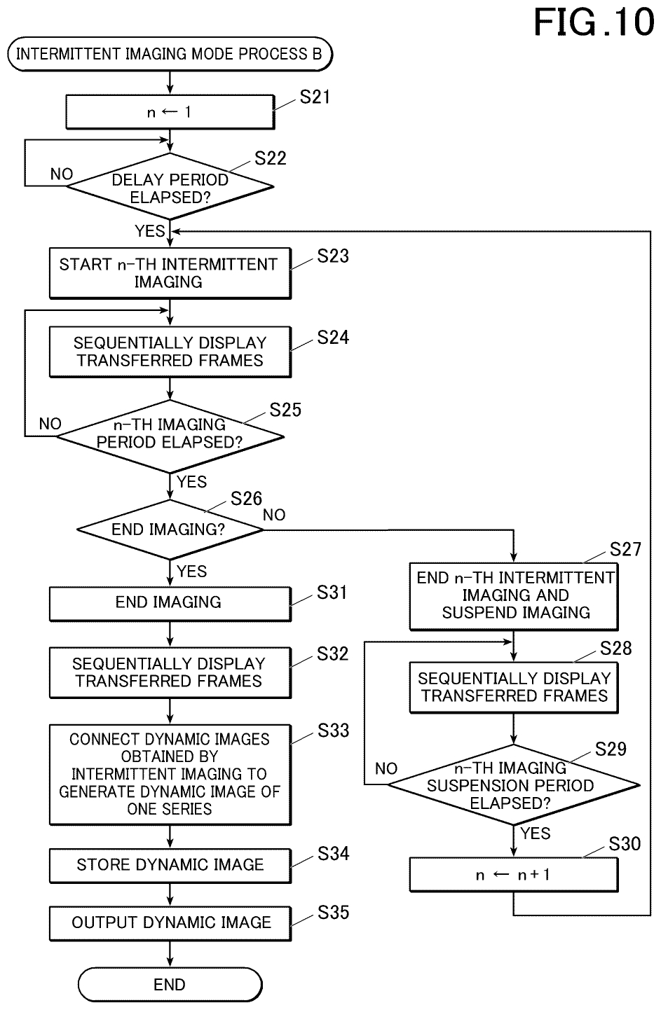

FIG. 10 is a flowchart of the intermittent imaging mode process B in the present embodiment. The intermittent imaging mode process B is executed by the controller 101 in cooperation with a program stored in the storage section 104.

In the intermittent imaging mode process B, first, the controller 101 sets a variable n to 1 (step S21).

Next, the controller 101 waits until a specified delay period elapses (step S22). When the designated delay period has elapsed (step S22; YES), the controller 101 causes the drive section 106 to cause the radiation source 2 to start pulse irradiation, thereby starting the n-th intermittent imaging (step S23).

For example, the controller 101 causes the drive section 106 and the radiation source 2 to perform pulse irradiation at a frame rate specified in the irradiation conditions.

When the FPD 3 detects radiation, the FPD 3 repeats a process for obtaining a frame with an accumulation time and a reading time corresponding to a frame rate specified in the image reading conditions. The FPD 3 adds, to the obtained frame, the frame number indicating the imaging order and the identification number of the intermittent imaging and transfers the frame to the main body 1.

The controller 101 receives, via the first communication section 105a, the frames transferred from the FPD 3 and sequentially displays the frames in the image display section 13b (step S24).

Next, the controller 101 determines whether the imaging period for the n-th intermittent imaging has elapsed or not (step S25).

When determining that the imaging period for the n-th intermittent imaging has not elapsed (step S25; NO), the controller 101 returns to step S24. That is, the controller 101 continues the n-th intermittent imaging, receives frames transferred from the FPD 3 via the first communication section 105a, and sequentially displays the frames in the image display section 13b.

On the other hand, when determining that the imaging period for the n-th intermittent imaging has elapsed (step S25; YES), the controller 101 determines whether to end imaging (step S26).

If the imaging suspension period or the imaging period after the n-th intermittent imaging is not specified, the controller 101 determines to end imaging.

When determining not to end imaging (step S26; NO), the controller 101 ends the n-th intermittent imaging and suspends the imaging (step S27).

That is, the controller 101 causes the drive section 106 to suspend (temporarily stop) irradiation by the radiation source 2. When radiation is not detected, the FPD 3 transmits untransmitted frames while waiting for the next detection of radiation. While radiation is not detected, the FPD 3 may continue accumulation and reading but discards images obtained during this period and does not transfer the images to the main body 1.

The controller 101 keeps receiving the frames forwarded from the FPD 3 via the first communication section 105a and causes the image display section 13b to sequentially display the frames (step S28).

Next, the controller 101 determines whether the n-th imaging suspension period has elapsed or not (step S29).

When determining that the n-th imaging suspension period has not elapsed (step S29; NO), the controller 101 returns to step S28.

When determining that the n-th imaging suspension period has elapsed (step S29; YES), the controller 101 increments the variable n (step S30) and returns to step S23. That is, the controller 101 starts the next intermittent imaging and sequentially displays frames obtained by the imaging in the image display section 13b.

In Step S26, when determining to end imaging (Step S26; YES), the controller 101 instructs the drive section 106 and the FPD 3 to end the imaging and ends the imaging (Step S31).

The controller 101 keeps receiving the frames forwarded from the FPD 3 via the first communication section 105a and causes the image display section 13b to sequentially display the frames (step S32).

After all the frames obtained by the imaging are displayed, the controller 101 connects the multiple dynamic images obtained by multiple times of intermittent imaging to generate a dynamic image of one series (step S33).

Since the process of step S33 is the same as step S14 of FIG. 6, the description thereof is omitted.

Next, the controller 101 associates the generated dynamic image with the supplementary information and stores the dynamic image in the image storage region of the storage section 104 (step S34). The controller 101 also transmits (outputs) the generated dynamic image to the PACS 40 and/or the analysis device 50 via the second communication section 105b (step S35). The controller 101 then ends the intermittent imaging mode process B.

As described in the first and second embodiments, the dynamic imaging system 10 can perform dynamic imaging multiple times during the period between one time of imaging start and one time of imaging end, based on the imaging period and the imaging suspension period specified by the user. Therefore, the dynamic imaging system 10 can obtain dynamic images for a necessary period while suspending imaging in a period for which imaging is not required to suppress an increase of the exposure dose.

For example, in a case where dynamic imaging is performed to check a treatment such as catheter insertion, it is necessary to check the start of the catheter insertion and the result of the catheter insertion. In conventional dynamic imaging, the result of the catheter insertion cannot be imaged if the insertion takes a long time. On the other hand, in the intermittent imaging mode, the dynamic imaging system 10 does not perform imaging for part of the treatment that need not be imaged. Therefore, the result of the treatment can be imaged.

Hereinafter, modification examples of the above-described embodiments are described.

### Modification Example 1

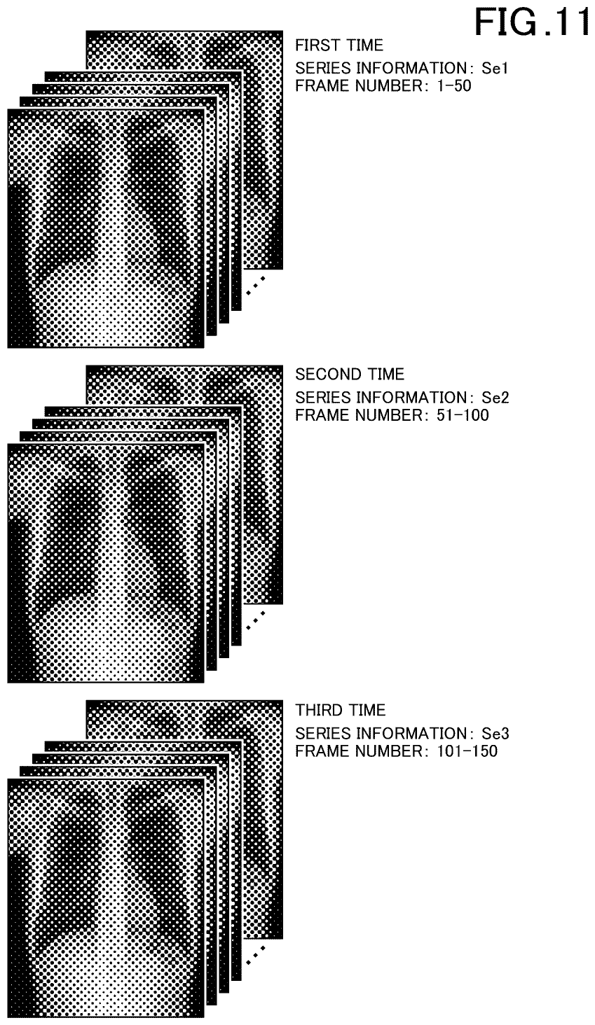

In the first and second embodiments, the controller 101 connects multiple dynamic images obtained by imaging in the intermittent imaging mode. Instead, the controller 101 may generate each dynamic image obtained by each time of intermittent imaging as a dynamic image of one series. For example, consider a case where: frames of a dynamic image in the first intermittent imaging have frame numbers 1 to 50; frames of a dynamic image in the second intermittent imaging have frame numbers 51 to 100; and frames of a dynamic image in the third intermittent imaging have frame numbers 101 to 150. In such a case, for example, as illustrated in FIG. 11, the controller 101 attaches series information Se1 to the frames having the frame numbers 1 to 50. The controller 101 attaches series information Se2 to the frames having the frame numbers 51 to 100. The controller 101 attaches series information Se3 to the frames having the frame numbers 101 to 150. For the frames having the series information Se2 and the frames having the series information Se3, the controller 101 assigns the frame number 1 to the frame having the smallest frame number and reassigns frame numbers to the subsequent frames. In addition to the series information, the controller 101 adds a frame number, patient information, and examination information to each of the frames.

At the end of each series of dynamic image, a summary image containing imaging conditions of the series may be inserted.

It is preferable that imaging time be included in information added to each frame. With the imaging time, it is possible to recognize from what time until what time each dynamic image of intermittent imaging has been obtained. Accordingly, the imaging period of each intermittent imaging can be included in the summary image. For example, in checking the process of inserting the catheter by intermittent imaging, it is preferable to attach the imaging time to each frame in order to grasp the time required for the insertion. Thus, the user is allowed to grasp to what extent the catheter is inserted with how much time.

Furthermore, at the main body 1, the user may be allowed to select a series to be transmitted to the PACS 40 and/or the analysis device 50. For example, the controller 101 displays a series selection button on the imaging screen 130. The controller 101 displays the dynamic image of the series selected by the selection button on the image display section 13b. In this state, when the output button 13e is pressed, the controller 101 transmits (outputs) the dynamic image of the selected series to the PACS 40 and/or the analysis device 50 via the second communication section 105b. When the imaging failure button 13f is pressed, the controller 101 discards the dynamic image of the selected series without transmitting the dynamic image.

The analysis device 50 may perform different analyses for the respective times of intermittent imaging. For example, assume a case where a contrast medium is used to image the swallowing motion. By applying different processes for the respective times of intermittent imaging, the intensity of image processing and the analysis process can be differentiated, depending on where (which site) the contrast medium is passing.

Thus, according to the modification example 1, it is possible to handle dynamic images obtained in the respective times of intermittent imaging in the intermittent imaging mode as dynamic images of different series.

The user may be allowed to select whether the multiple dynamic images obtained in the intermittent imaging mode are connected as one dynamic image or separated as different series.

### Modification Example 2

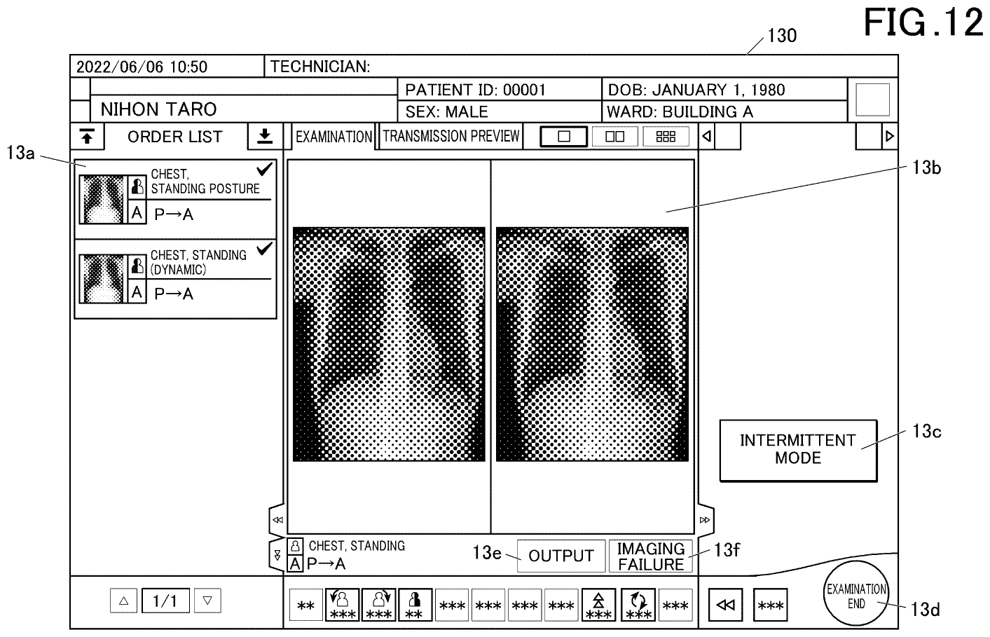

In displaying the transferred dynamic image, the image display section 13b may be divided into multiple areas so that the transferred image can be displayed next to another image so as to be compared with each other, as illustrated in FIG. 12. For example, the controller 101 divides the image display section 13b into right and left sections and displays the frame of the currently performed n-th intermittent imaging in the right section R and the frame of the (n−1)th intermittent imaging in the left section L. Instead, the controller 101 may display the frame of currently performed intermittent imaging in one section of the image display section 13b and a reference image in a different section. The reference image is, for example, a still image or a key frame (representative frame) of a moving image obtained in the same examination. For example, if the catheter insertion is imaged in the current dynamic imaging, a still image of the same site obtained before the catheter insertion is displayed as the reference image. The user can select the reference image from medical images stored in the storage section 104.

As described above, according to the dynamic imaging system 10, the controller 101 of the main body 1 controls the radiation source 2 and the FPD 3 to operate in an intermittent imaging mode in which dynamic imaging is performed multiple times from one time of imaging start to one time of imaging end, based on a specified imaging period and a specified imaging suspension period.

Since the dynamic imaging system 10 suspends dynamic imaging when imaging is not necessary, the dynamic imaging system 10 can obtain a dynamic image for a necessary period while suppressing an increase of the exposure dose.

Further, the controller 101 associates the multiple dynamic images, which are obtained by the multiple times of dynamic imaging in the intermittent imaging mode, with each other and outputs the associated dynamic images via the second communication section 105b. For example, the controller 101 connects the multiple dynamic images to generate one dynamic image of one series and outputs the one dynamic image via the second communication section 105b. In particular, the controller 101 outputs the multiple dynamic images as a dynamic image of an identical series in an identical examination via the second communication section 105b. Thus, it is possible to manage multiple dynamic images obtained in multiple times of imaging in the intermittent imaging mode as a dynamic image of one series. For example, multiple dynamic images obtained by multiple times of imaging in the intermittent imaging mode can be managed as a dynamic image of the same series in the same examination.

Further, the controller 101 adds, to the one dynamic image, information for identifying a range of frames constituting each of the multiple dynamic images. Therefore, it is possible to identify each of the multiple dynamic images obtained by multiple times of imaging in the intermittent imaging mode.

Further, the controller 101 outputs the multiple dynamic images, which are obtained by the multiple times of dynamic imaging in the intermittent imaging mode, as dynamic images of different series in an identical examination via the second communication section 105b. Therefore, it is possible to manage multiple dynamic images obtained by multiple times of imaging in the intermittent imaging mode as dynamic images of different series in the same examination.

Further, the imaging period and the imaging suspension period in the intermittent imaging mode can be specified by manipulation of the exposure switch 102a that is configured for instructing the radiation source 2 to emit radiation. Therefore, the user can specify the imaging period and the imaging suspension period during imaging.

Further, the imaging period and the imaging suspension period in the intermittent imaging mode can be specified on the specifying screen 131 shown on the display part 103. Therefore, the user can specify the imaging period and the imaging suspension period before imaging.

Further, the controller 101 causes the display part 103 to sequentially display frames of the multiple dynamic images obtained by the multiple times of dynamic imaging in the intermittent imaging mode. Therefore, the user can check the frames of multiple dynamic images obtained by multiple times of imaging in the intermittent imaging mode.

For example, the display part 103 displays frames of one dynamic image among the multiple dynamic images next to frames of another dynamic image that has been obtained earlier. Therefore, the user can check frames of one of the multiple dynamic images obtained in the intermittent imaging mode in comparison with frames of another dynamic image that has been obtained earlier.

Further, for example, the display part 103 displays frames of the multiple dynamic images next to a reference image selected beforehand. Therefore, the user can check the frames of the multiple dynamic images in comparison with the reference image.

The controller 101 performs control such that a total dose of radiation emitted by the radiation source 2 in the multiple times of dynamic imaging from the start to the end of imaging in the intermittent imaging mode does not exceed a predetermined upper limit value. Therefore, it is possible to obtain multiple dynamic images while suppressing the exposure dose.

The above-described embodiments are preferred examples of the present invention and not intended to limit the present invention.

For example, although the present invention is applied to the dynamic imaging system 10 configured to perform round-visit imaging in the above-described embodiments, the present invention may be applied to a dynamic imaging system configured to perform imaging in an imaging room.

In the above embodiment, the dynamic imaging is described as obtaining multiple images by irradiating a subject with pulsed radiation such as X-rays. However, multiple images may be obtained by continuously irradiating the subject with radiation such as X-rays at a low dose rate (continuous irradiation).

Although a hard disk, a semiconductor nonvolatile memory, or the like is used in the above description as a computer-readable medium storing the program according to the present invention, the present invention is not limited to this example. As other computer-readable media, portable recording media such as CD-ROMs can be applied. In addition, a carrier wave is also applied as a medium for providing data of the program according to the present invention via a communication line.

The detailed configuration and the detailed operation of the dynamic imaging system can be appropriately changed without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. A dynamic imaging system comprising:
a radiation source;
a radiation detector; and
a hardware processor, wherein
the dynamic imaging system is configured to perform dynamic imaging of obtaining a dynamic image constituted of multiple frames by irradiating a subject with radiation by the radiation source and detecting the radiation passing through the subject by the radiation detector; and
the hardware processor controls the radiation source and the radiation detector to operate in an intermittent imaging mode in which dynamic imaging is performed multiple times from one time of imaging start to one time of imaging end, based on a specified imaging period and a specified imaging suspension period.

2. The dynamic imaging system according to claim 1, wherein
the hardware processor associates multiple dynamic images obtained by the multiple times of dynamic imaging in the intermittent imaging mode with each other and outputs the associated dynamic images via an outputter.

3. The dynamic imaging system according to claim 2, wherein
the hardware processor connects the multiple dynamic images to generate one dynamic image of one series and outputs the one dynamic image via the outputter.

4. The dynamic imaging system according to claim 2, wherein
the hardware processor outputs the multiple dynamic images as dynamic images of an identical series in an identical examination via the outputter.

5. The dynamic imaging system according to claim 3, wherein
the hardware processor adds, to the one dynamic image, information for identifying a range of frames constituting each of the multiple dynamic images.

6. The dynamic imaging system according to claim 1, wherein
the hardware processor outputs multiple dynamic images obtained by the multiple times of dynamic imaging in the intermittent imaging mode as dynamic images of different series in an identical examination via the outputter.

7. The dynamic imaging system according to claim 1, wherein
the imaging period and the imaging suspension period are specified by manipulation of an exposure switch that is configured for instructing the radiation source to emit radiation.

8. The dynamic imaging system according to claim 1, further comprising a display, wherein
the imaging period and the imaging suspension period are specified based on inputs on a specifying screen shown on the display, the specifying screen being configured for specifying the imaging period and the imaging suspension period.

9. The dynamic imaging system according to claim 1, further comprising a display configured to sequentially display frames of multiple dynamic images obtained by the multiple times of dynamic imaging in the intermittent imaging mode.

10. The dynamic imaging system according to claim 9, wherein
the display displays frames of one dynamic image among the multiple dynamic images next to a frame of another dynamic image having been obtained.

11. The dynamic imaging system according to claim 9, wherein
the display displays frames of the multiple dynamic images next to a reference image selected beforehand.

12. The dynamic imaging system according to claim 1, wherein the hardware processor performs control such that a total dose of radiation emitted by the radiation source in the multiple times of dynamic imaging does not exceed a predetermined upper limit value.

13. An imaging control method for a dynamic imaging system that includes a radiation source and a radiation detector and that is configured to perform dynamic imaging of obtaining a dynamic image constituted of multiple frames by irradiating a subject with radiation by the radiation source and detecting the radiation passing through the subject by the radiation detector, the method comprising:
controlling the radiation source and the radiation detector to operate in an intermittent imaging mode in which dynamic imaging is performed multiple times from one time of imaging start to one time of imaging end, based on a specified imaging period and a specified imaging suspension period.

14. A non-transitory computer-readable storage medium storing a program for a computer of a dynamic imaging system that includes a radiation source and a radiation detector and that is configured to perform dynamic imaging of obtaining a dynamic image constituted of multiple frames by irradiating a subject with radiation by the radiation source and detecting the radiation passing through the subject by the radiation detector, the program causing the computer to:
control the radiation source and the radiation detector to operate in an intermittent imaging mode in which dynamic imaging is performed multiple times from one time of imaging start to one time of imaging end, based on a specified imaging period and a specified imaging suspension period.
